(19) United States
(12) Patent Application Publication
(10) Pub. No.: US 2025/0098989 A1
(43) Pub. Date: Mar. 27, 2025

(54) CONTINUOUS BLOOD GLUCOSE MONITORING SYSTEM

(71) Applicant: Shenzhen Goodix Technology Co., Ltd., Shenzhen (CN)

(72) Inventors: Shuqing CHENG, Shenzhen City (CN); Haifeng LIU, Shenzhen (CN)

(21) Appl. No.: 18/824,033

(22) Filed: Sep. 4, 2024

(30) Foreign Application Priority Data
Sep. 21, 2023 (CN) 202322583259.0

(51) Int. Cl. A61B 5/145; A61B 90/00

(52) U.S. Cl. CPC A61B 5/14503; A61B 5/14532; A61B 2090/064; A61B 2560/063; A61B 2562/166

(57) ABSTRACT

Provided is a continuous blood glucose monitoring system, including a container shell, and a spring, a continuous blood glucose detection apparatus and a trigger structure which are arranged inside the container shell. One end of the spring is connected to an interior of the container shell, and the other end of the spring is connected to the continuous blood glucose detection apparatus. The trigger structure is arranged on a side edge of the interior of the container shell, a protruding portion is arranged on the trigger structure, which is unlockably locked with the continuous blood glucose detection apparatus. The continuous blood glucose detection apparatus includes: an apparatus shell and a circuit board arranged in the apparatus shell; a probe assembly being connected to the circuit board; and a pressure detection sensor connected to the circuit board, which being configured to detect a pressure change signal on the apparatus shell.

### TECHNICAL FIELD

The present application relates to the technical field of blood glucose detection, and in particular to a continuous blood glucose monitoring system.

### BACKGROUND

A continuous glucose monitor (CGM) is a blood glucose monitoring system including a microelectrode biosensor, a signal transmitter and a signal receiver, and can continuously monitor blood glucose for a long time (such as 7 days, 14 days or as long as 21 days). As an electronic medical device capable of being worn for a long time, CGM puts forwards higher requirements for miniaturization and low power consumption so as to achieve the purposes of convenient use, simple operation and wearing comfortability. Furthermore, CGM is in a power-off or low-power-consumption sleep mode when in a shelf life. Once CGM is implanted into a human body, the blood glucose detection function of CGM and the data function begin to work, but a conventional physical switch is generally used to control the on and off of the CGM.

Therefore, how to automatically switch on and off the blood glucose detection function of CGM to facilitate the miniaturization design of CGM and be convenient for users to use is an urgent technical problem to be solved.

### SUMMARY

A continuous blood glucose monitoring system provided by a first aspect of the embodiments of the present application includes:
a container shell, and a spring, a continuous blood glucose detection apparatus and a trigger structure which are arranged in an interior of the container shell, where
one end of the spring is connected to the interior of the container shell, and the other end of the spring is connected to the continuous blood glucose detection apparatus;
the trigger structure is arranged on a side edge of the interior of the container shell, a protruding portion is arranged on the trigger structure, and the protruding portion is unlockably locked with the continuous blood glucose detection apparatus; and
the continuous blood glucose detection apparatus includes:
an apparatus shell and a circuit board arranged in the apparatus shell, an electronic component being carried on the circuit board;
a probe assembly for penetrating skin, the probe assembly being connected to the circuit board; and
a pressure detection sensor connected to the circuit board, the pressure detection sensor being configured to detect a pressure change signal on the apparatus shell.

In a possible embodiment, the trigger structure is rotatably arranged on the side edge of the interior of the container shell.

In a possible embodiment, the probe assembly includes a probe, and the probe extends to the outside through an opening at one end of the apparatus shell.

In a possible embodiment, the pressure detection sensor is arranged in an interior of the apparatus shell and is electrically connected to the circuit board.

In a possible embodiment, the pressure detection sensor is arranged on an upper surface of an interior of the apparatus shell.

In a possible embodiment, the pressure detection sensor is arranged on a lower surface of the interior of the apparatus shell.

In a possible embodiment, the pressure detection sensor is arranged on a side edge of the interior of the apparatus shell.

In a possible embodiment, the circuit board is a printed circuit board.

In a possible embodiment, the electronic component includes a control unit, and the control unit is configured to control the on and off of the function of the continuous blood glucose monitoring system.

In a possible embodiment, the pressure detection sensor is a capacitive pressure sensor.

In a possible embodiment, the pressure detection sensor is a resistive pressure sensor.

The embodiments of the present application provide a continuous blood glucose monitoring system. The continuous blood glucose monitoring system can identify the pressure change on the apparatus shell, thereby automatically switching on and off a blood glucose detection function.

### BRIEF DESCRIPTION OF DRAWINGS

FIG. 1 is a schematic structural diagram of a continuous blood glucose monitoring system according to an embodiment of the present application; and

FIG. 2 is a schematic diagram of a working state of a continuous blood glucose monitoring system according to an embodiment of the present application.

### DESCRIPTION OF THE EMBODIMENTS

The technical solutions in the embodiments of the present application will be clearly and completely described below with reference to the accompanying drawings.

The terms used in the present application are merely for describing specific embodiments, rather than limiting the present application. The terms “a/an” and “the” in the singular form used in the present application and the appended claims are also intended to include the plural forms, unless otherwise clearly indicated in the context.

In addition, the terms “first” and “second” are merely used to describe similar objects and cannot be interpreted as indicating or implying relative importance or implicitly indicating the number of technical features indicated. Thus, a feature defined as “first” or “second” may explicitly or implicitly include one or more such features.

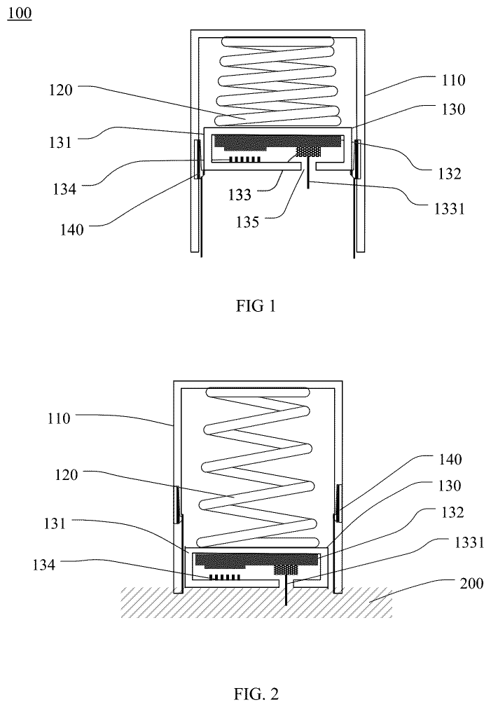

FIG. 1 is a schematic structural diagram of a continuous blood glucose monitoring system 100 according to an embodiment of the present application.

As shown in FIG. 1, the continuous blood glucose monitoring system 100 includes: a container shell 110, and a spring 120, a continuous blood glucose detection apparatus 130 and a trigger structure 140 which are arranged in an interior of the container shell 110.

One end of the spring 120 is connected to an interior of the container shell 110, and the other end of the spring 120 is connected to the continuous blood glucose detection apparatus 130.

The trigger structure 140 is arranged on a side edge of the interior of the container shell 110, a protruding portion is arranged on the trigger structure 140, and the protruding portion is unlockably locked with the continuous blood glucose detection apparatus 130.

The continuous blood glucose detection apparatus 130 includes:
an apparatus shell 131 and a circuit board 132 arranged in the apparatus shell 131, an electronic component being carried on the circuit board 132;
a probe assembly 133 for penetrating skin, the probe assembly 133 being connected to the circuit board 132; and
a pressure detection sensor 134 connected to the circuit board 132, the pressure detection sensor 134 being configured to detect a pressure change signal on the apparatus shell 131.

The continuous blood glucose monitoring system 100 provided by the present application can identify the pressure change on the apparatus shell 131, thereby automatically switching on and off a blood glucose detection function.

In an optional embodiment, the trigger structure 140 is rotatably arranged on the side edge of the interior of the container shell 110. When the trigger structure 140 rotates by a certain angle, the trigger structure 140 can be changed from an untriggered state to a triggered state. A rotatable trigger structure 140 like this is simple in structure and easy to implement.

Optionally, the circuit board 132 may be a printed circuit board (PCB); and the printed circuit board not only may be electrically connected to the probe assembly 133 and the pressure detection sensor 134, but also may play a certain supporting role for the probe assembly 133 and the pressure detection sensor 134.

In an optional embodiment, the electronic component carried on the circuit board 132 includes a control unit (CU), and the control unit is configured to control the on and off of the function of the continuous blood glucose monitoring system. After the pressure detection sensor 134 detects that the pressure on the apparatus shell 131 becomes lower or disappears, pressure change information may be transmitted to the control unit, and the control unit can determine whether to switch on a blood glucose detection function and a data transmission function according to the received pressure change information, thereby automatically switching on and off the blood glucose detection function of the continuous blood glucose monitoring system 100.

Optionally, referring to FIG. 1, the probe assembly 133 includes a probe 1331, and the probe 1331 extends to the outside through an opening 135 at one end of the apparatus shell 131, so that the probe 1331 can be conveniently implanted below skin.

Optionally, the pressure detection sensor 134 is arranged in the apparatus shell 131 and is electrically connected to the circuit board 132. The apparatus shell 131 may provide a certain supporting and protection effect for the pressure detection sensor 134 arranged in the interior.

Optionally, an electronic component carried on the circuit board 132 further includes a pressure detection chip, and the pressure detection sensor 134 may be connected to the pressure detection chip on the circuit board 132 through a lead.

Optionally, the pressure detection sensor 134 is a capacitive pressure sensor, and the capacitive pressure sensor may monitor the pressure change in real time by measuring the change of a capacitance value.

Optionally, the pressure detection sensor 134 is a resistive pressure sensor, and the resistive pressure sensor may monitor the pressure change in real time by measuring the change of a resistance value.

As shown in FIG. 1, when the trigger structure 140 of the continuous blood glucose monitoring system 100 is not triggered, the continuous blood glucose detection apparatus 130 is locked by the trigger structure 140, and the spring 120 above the continuous blood glucose detection apparatus 130 is in a compressed state.

At this time, the apparatus shell 131 of the continuous blood glucose detection apparatus 130 is deformed due to a pressure exerted by the spring 120. The deformation may be detected by the pressure detection sensor 134 arranged inside the blood glucose detection apparatus 130 and configured to detect the pressure change, then the pressure detection sensor 134 transmits the detected pressure signal to the control unit on the circuit board 132, and the control unit can determine that the continuous blood glucose monitoring system 100 is not working at this time according to the received pressure signal, and can control the blood glucose detection and data transmission functions of the continuous blood glucose monitoring system 100 to be in an off or sleep state, thereby reducing the power consumption of the continuous blood glucose monitoring system 100.

In an optional embodiment, the pressure detection sensor 134 is arranged on an upper surface of an interior of the apparatus shell 131. An upper half portion of the apparatus shell 131 is in direct contact with the spring 120, and is deformed most obviously due to the pressure exerted by the spring 120. Therefore, the pressure detection sensor 134 is arranged on the upper surface of the interior of the apparatus shell 131, and the pressure detection sensor 134 can detect the pressure change more easily, thereby improving the pressure detection accuracy.

As another optional embodiment, the pressure detection sensor 134 is arranged on a lower surface of the interior of the apparatus shell 131. The pressure detection sensor 134 and the probe assembly 133 are both located in a space formed between the circuit board 132 and a lower half portion of the apparatus shell 131. Such design can reduce the thickness of the continuous blood glucose monitoring system 100, thereby improving the use experience of users.

As another optional embodiment, the pressure detection sensor 134 is arranged on a side edge of the interior of the apparatus shell 131. Preferably, the pressure detection sensor 134 is arranged near a part of the apparatus shell 131 in contact with the trigger structure 140. In the embodiments of the present application, the trigger structure 140 is arranged on a side edge of the interior of the container shell 110, a protruding portion is arranged on the trigger structure 140, and the protruding portion is unlockably locked with the continuous blood glucose detection apparatus 130. Therefore, the pressure detection sensor 134 is arranged on the side edge of the interior of the apparatus shell 131, and can detect the pressure change of the contact part of the trigger structure 140 and the apparatus shell 131, thereby controlling the activation and shutdown of the blood glucose and data transmission functions of the whole continuous blood glucose monitoring system 100, and improving the use experience of users.

FIG. 2 is a schematic diagram of a working state of a continuous blood glucose monitoring system 100 according to an embodiment of the present application.

As shown in FIG. 2, when a user needs to use the continuous blood glucose monitoring system 100, the trigger structure 140 of the continuous blood glucose monitoring system 100 is triggered, the continuous blood glucose detection apparatus 130 and the trigger structure 140 are unlocked, the spring 120 above the continuous blood glucose detection apparatus 130 is changed from a compressed state to an extended state and pushes the continuous blood glucose detection apparatus 130 to move downward, the continuous blood glucose detection apparatus 130 is pushed to a surface of human skin 200, and the probe assembly 133 with a puncture function or a puncture functional accessory is implanted into the human skin 200.

At this time, a pressure applied by the spring 120 to the apparatus shell 131 of the continuous blood glucose detection apparatus 130 is reduced or disappears. The pressure change may be detected by the pressure detection sensor 134 arranged inside the continuous blood glucose detection apparatus 130 and configured to detect the pressure change, then the pressure detection sensor 134 transmits the detected pressure change signal to the control unit on the circuit board 132, and the control unit can determine that the continuous blood glucose monitoring system 100 is in a working state at this time according to the received pressure change signal, and can control the blood glucose detection and data transmission functions of the continuous blood glucose monitoring system 100 to be in an on or activated state, so that the power consumption of the continuous blood glucose monitoring system 100 can work normally.

In the embodiments of the present application, the continuous blood glucose monitoring system 100 can identify the pressure change on the apparatus shell 131, and automatically switch on and off the blood blood glucose detection function, thereby facilitating the miniaturization design of the continuous blood glucose monitoring system and being convenient for customers to use.

The preferred embodiments of the present application are described in detail above with reference to the drawings. However, the present application is not limited to the specific details of the above embodiments, various simple variations may be made to the technical solutions of the present application within the scope of the technical concept of the present application, and these simple variations fall within the protection scope of the present application.

1. A continuous blood glucose monitoring system, comprising:
a container shell, and a spring, a continuous blood glucose detection apparatus and a trigger structure which are arranged in an interior of the container shell, wherein
one end of the spring is connected to an interior of the container shell, and the other end of the spring is connected to the continuous blood glucose detection apparatus;
the trigger structure is arranged on a side edge of the interior of the container shell, a protruding portion is arranged on the trigger structure, and the protruding portion is unlockably locked with the continuous blood glucose detection apparatus; and
the continuous blood glucose detection apparatus comprises:
an apparatus shell and a circuit board arranged in the apparatus shell, an electronic component being carried on the circuit board;
a probe assembly for penetrating skin, the probe assembly being connected to the circuit board; and
a pressure detection sensor connected to the circuit board, the pressure detection sensor being configured to detect a pressure change signal on the apparatus shell.

2. The continuous blood glucose monitoring system according to claim 1, wherein the trigger structure is rotatably arranged on the side edge of the interior of the container shell.

3. The continuous blood glucose monitoring system according to claim 1, wherein the probe assembly comprises a probe, the probe extending to the outside through an opening at one end of the apparatus shell.

4. The continuous blood glucose monitoring system according to claim 1, wherein the pressure detection sensor is arranged in the interior of the apparatus shell and is electrically connected to the circuit board.

5. The continuous blood glucose monitoring system according to claim 4, wherein the pressure detection sensor is arranged on an upper surface of an interior of the apparatus shell.

6. The continuous blood glucose monitoring system according to claim 4, wherein the pressure detection sensor is arranged on a lower surface of the interior of the apparatus shell.

7. The continuous blood glucose monitoring system according to claim 4, wherein the pressure detection sensor is arranged on a side edge of the interior of the apparatus shell.

8. The continuous blood glucose monitoring system according to claim 1, wherein the circuit board is a printed circuit board.

9. The continuous blood glucose monitoring system according to claim 1, wherein the electronic component comprises a control unit, the control unit being configured to control the on and off of the function of the continuous blood glucose monitoring system.

10. The continuous blood glucose monitoring system according to claim 1, wherein the pressure detection sensor is a capacitive pressure sensor or a resistive pressure sensor.
